(19) United States
(12) Patent Application Publication
(10) Pub. No.: US 2009/0259295 A1
(43) Pub. Date: Oct. 15, 2009

(54) Method of Delivering Graft Material Without the Use of a Stent

(75) Inventor: Matthew Rust, North Vancouver (CA)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/102,336

(22) Filed: Apr. 14, 2008

(51) Int. Cl. A61F 2/06

(52) U.S. Cl. 623/1.23; 606/108; 128/898

(57) ABSTRACT

A method of delivering a tubular endoluminal graft through a body lumen in which the graft is expanded and temporarily held in place by an expandable support structure at the distal end of a graft delivery system until staples from an endovascular stapling device can be delivered to permanently secure the graft to the vessel wall. The support structure radially expands to radially expand the graft against a vessel wall. The support structure holds the graft in place while a stapling device is delivered between the outer shaft and the radially expanded graft. The stapling device delivers one or more staples for attaching the graft to the vessel wall. Once the graft is attached to the vessel wall, it is disconnected from the support structure so that the support structure may be radially contracted and the delivery system may be removed.

### FIELD OF THE INVENTION

The present invention relates generally to methods and systems for delivering a graft through a body lumen for the treatment of vascular disease. More particularly, the present invention relates to a graft delivery system for use in conjunction with a medical stapling system to fix graft material to a vessel wall.

### BACKGROUND OF THE INVENTION

In modern medical practice, it is sometimes desirable to pass a stapling device into or through the wall of a luminal anatomical structure (e.g., a blood vessel or other anatomical conduit) for the purpose of attaching an article (e.g., an endoluminal graft) or other apparatus to the wall of the anatomical structure. Such endovascular stapling devices have shown effectiveness in preventing undesired graft migration.

Grafting procedures have been used to treat aneurysms, such as aneurysms of the abdominal aorta and of the descending thoracic aorta. Aneurysms result from weak blood vessel walls which can balloon due to aging and disease and pressure in the vessel. In addition, aneurysmal vessels have a potential to rupture, causing internal bleeding and potentially life threatening conditions. Grafts are used to isolate aneurysms or other blood vessel abnormalities from normal blood pressure, reducing pressure on the weakened vessel wall and reducing the chance of vessel rupture. A tubular endovascular graft is placed within the aneurysmal blood vessel to create a new flow path and an artificial flow conduit through the aneurysm, thereby reducing if not nearly eliminating the exertion of blood pressure on the aneurysm. The graft typically incorporates or is combined with one or more radially expandable stent(s) to be radially expanded in situ to anchor the tubular graft to the wall of the blood vessel at sites upstream and downstream of the aneurysm. Thus, endovascular grafts are typically held in place by mechanical engagement and friction because of the force of the self-expanding or balloon expandable stents. Hooks or barbs have also been used to affix or help affix grafts to vessels.

However, the stent(s) support structure may fail to establish an acceptable long term fixation with the blood vessel wall. In such an event, the graft may undergo undesirable migration or slippage, or blood may leak into the aneurysmal sac (sometimes referred to as an “endoleak”). Further, including a stent structure with the graft material increases the thickness of the wall of the device and its bulk when compressed. Delivering such a bulky device which makes a catheter containing the stent graft have a larger crossing is difficult when the vasculature access diameter is small. Thus, to reduce the delivery catheter crossing profile it may be desirable to secure graft material endovascularly without the use of a radially expandable stent. As previously mentioned, stapling devices have shown effectiveness for securing the graft to the vessel wall. However, the graft material must be delivered to the treatment site, expanded or deployed, and temporarily held in place until staples from an endovascular stapling device can be delivered to secure the graft to the vessel wall. Thus, a need exists in the art for the development of new delivery devices which may be used to deliver and secure an endoluminal graft to the surrounding wall of a blood vessel or other tubular anatomical conduit until a stapling device may be utilized to permanently attach the graft or other article.

### BRIEF SUMMARY OF THE INVENTION

A method of delivering a tubular endoluminal graft through a body lumen is presented. A graft delivery system is tracked through an artery to a target location within the lumen of a body vessel. The graft delivery system includes an expandable support structure at a distal portion of the system, wherein the graft is releasably attached to the support structure. The support structure is radially expanded to form a balloon shaped cage which may approximate ellipsoid, spherical, and/or cylinder like shapes in its expanded configuration such that at least a portion of the graft abuts the vessel wall. A stapling device is then tracked to the radially expanded graft to a position adjacent the target location within the body vessel. At least one staple is fired (delivered) from the stapling device to attach the graft to the vessel wall while the expanded support structure holds at least a portion of the graft against the vessel wall. The stapling device is retracted. The graft is disconnected from the support structure, and the support structure is radially contracted to a straightened delivery configuration. The graft delivery system is retracted with the support structure in the straightened delivery configuration. In one embodiment, the support structure is a plurality of strands having open spaces there between sufficient to allow the stapling device to maneuver through the support structure, the plurality of strands extending generally parallel to the blood flow. In another embodiment, the support structure is formed from a cage, e.g., a braided structure or mesh, having open spaces sufficient to allow the stapling device to maneuver through the support structure and deliver a staple without being obstructed by a wire of the cage.

Embodiments also relate to a graft delivery system for delivering a tubular endoluminal graft through a body lumen. The system includes an outer shaft and an inner shaft disposed within the lumen of the outer shaft. An expandable support structure is provided at a distal portion of the system, wherein a proximal end of the expandable support structure is connected to a distal end of the outer shaft and a distal end of the expandable support structure is connected to a distal end of the inner shaft. A graft is releasably attached to the support structure, the graft having no stent structure coupled thereto. An actuator is provided at a proximal portion of the system for moving the outer shaft relative to the inner shaft to expand the support structure to a configuration having open spaces sufficient to allow a stapling device to maneuver there through. In one embodiment of the present invention, the support structure is formed from a plurality of strands extending generally parallel to the blood flow. In another embodiment of the present invention, the support structure is formed from a mesh.

### BRIEF DESCRIPTION OF DRAWINGS

The foregoing and other features and advantages will be apparent from the following description as illustrated in the accompanying drawings. The accompanying drawings, which are incorporated herein and form a part of the specification, further serve to explain and to enable a person skilled in the pertinent art to make and use apparatus and methods described. The drawings are not to scale.

FIG. 1 is a schematic side view of a graft delivery system according to the present invention.

FIG. 2 is a cross-sectional view along line A-A of FIG. 1.

FIG. 3 is a schematic side view of the distal portion of the graft delivery system of FIG. 1, where the expandable support structure is in the unexpanded or straightened configuration.

FIG. 4 is a schematic side view of the distal portion of the graft delivery system of FIG. 1, where the expandable support structure is in the expanded or deployed configuration.

FIG. 5 is a schematic side view of the distal portion of another graft delivery system according to the present invention, where the expandable support structure is in the unexpanded or straightened configuration.

FIG. 6 is a schematic side view of the distal portion of the graft delivery system of FIG. 5, where the expandable support structure is in the expanded or deployed configuration.

FIGS. 7-14 illustrate, diagrammatically illustrate the steps of a method of implanting a graft within a blood vessel in accordance with the present invention.

### DETAILED DESCRIPTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to be limiting. Although the method and apparatus presented describes treatment in the context of blood vessels such as the aorta, the treatment may also be used in any other body passageways where it is deemed useful. Furthermore, there is no intention to be bound by any expressed or implied theory presented in the preceding technical field, background, brief summary or the following detailed description.

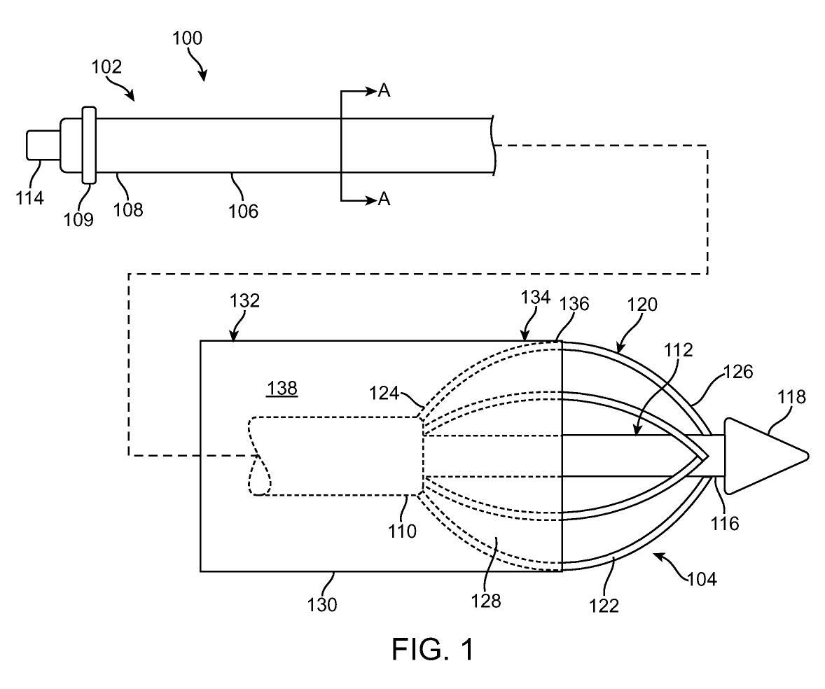

A method and system for delivering a graft includes a graft delivery system that has an expandable support structure at the distal end thereof for radially expanding to contact a vessel wall. The graft is temporarily held in place by the expanded support structure until staples from an endovascular stapling device can be delivered to permanently secure the graft to the vessel wall. Thus, the graft delivery system initially secures an endovascular graft without the use of a radially expandable stent. Utilizing staples to secure the graft improves long term patient care by avoiding possible migration of the graft. In addition, in the present system unlike prior stent graft systems which required a large enough catheter delivery profile to contain the compressed stiff metallic stent and adjacent fabric graft material, the delivery profile of the present graft delivery system is reduced and the flexibility of the graft delivery system is increased. Further details and description of embodiments are provided below with reference to FIGS. 1-14.

FIG. 1 is a schematic side view of graft delivery system 100, including a proximal portion 102 and a distal portion 104. Distal portion 104 includes an expandable support structure 120 for expanding a synthetic tubular graft 130. Graft 130 is preferably formed from a polyester fabric, such as DACRON, or other biocompatible material, such as PTFE (polytetrafluoroethylene). Graft delivery system 100 includes an outer shaft 106 having a proximal end 108 and a distal end 110, and an inner shaft 112 having a proximal end 114 and a distal end 116. Inner shaft 112 extends through the entire length of outer shaft 106 and support structure 120 to a distal tip 118 of system 100. Distal tip 118 may be flared and tapered to assist in tracking and securing graft 130 at the treatment site.

Outer shaft 106 is movable in an axial direction (i.e., along and relative to inner shaft 112) and extends to proximal portion 102 of system 100 where it may be controlled via a handle 109 to selectively expand graft 130 located at distal portion 104 of system 100. Expandable support structure 120 is located at distal portion 104 of system 100, and includes a plurality of strands or ribbons 122 that extend laterally while being oriented generally parallel to the blood flow when expanded. A proximal end 124 of support structure 120 is attached to distal end 110 of outer shaft 106, and a distal end 126 of support structure 120 is attached to distal end 116 of inner shaft 112. While holding proximal end 114 of inner shaft 112 fixed, outer shaft 106 may be distally advanced via handle 109 over inner shaft 112 (such that distal end 110 connected to support structure 120 also is distally advanced), while the attachment point between support structure 120 and inner shaft 112 remains fixed. As such, when outer shaft 106 is distally advanced, expandable support structure 120 radially expands to radially expand graft 130 which is releasably attached to the strands or ribbons of the support structure and press graft 130 against a vessel wall.

Support structure 120 may be attached to outer shaft 106 and inner shaft 112 in any suitable manner known in the art. For example, the connection may be formed by welding, such as by resistance welding, friction welding, laser welding or another form of welding such that no additional materials are used to connect support structure 120 to shafts 106, 112. Alternatively, support structure 120 can be connected to shafts 106, 112 by soldering, by the use of an adhesive, by the addition of a connecting element there between, or by another mechanical method.

As will be explained in more detail below, a stapling device may be delivered within space 138 between outer shaft 106 and radially expanded graft 130 for attaching graft 130 to a vessel wall of a body lumen. Embodiments of the present structure may be used with any conventional stapling device capable of securing graft 130 to a vessel wall. Thus, it will be apparent to those of ordinary skill in the art that any features of the stapling device discussed herein are exemplary in nature. For example, the stapling device may be any stapling device known in the art, including but not limited to those shown or described in US Patent Publication 20040176786 assigned to Edrich Vascular, US Patent Publication 20070073389 assigned to Aptus Endosystems, Inc., and US Patent Publication 20070162053 assigned to Anson Medical. A conventional stapling typically has a profile or an outer diameter of approximately 4 mm-5 mm (12-15 French units) and thus space 138 between the outside of the outer shaft 106 and the inside of the graft material 130 should be of a slightly larger size to ensure that a conventional stapling device can be advanced there through. Thus, it is desirable to minimize the delivery profile of system 100 as much as possible such that graft delivery system 100 may fit within relatively small vessels.

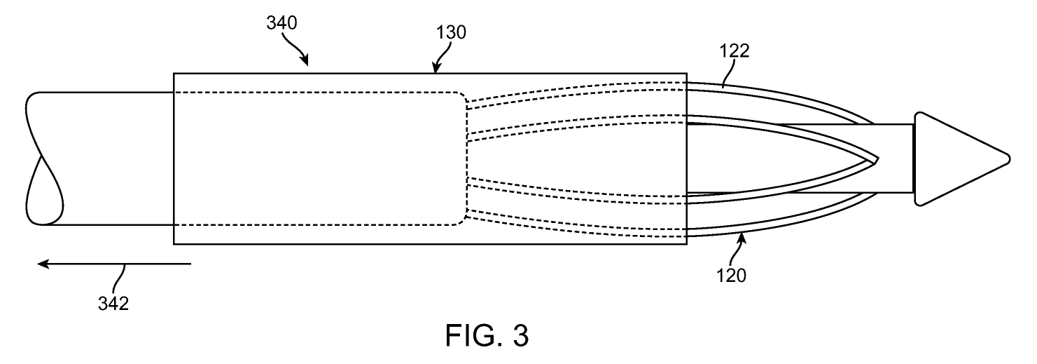

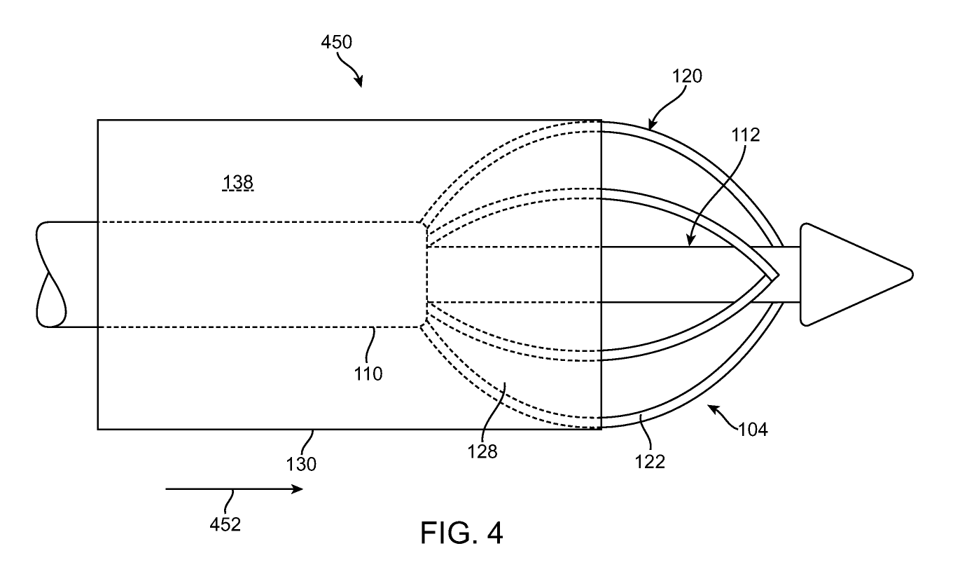

Strands or ribbons 122 of support structure 120 are approximately equal in length, extend generally parallel to the blood flow, and are uniformly circumferentially arranged about a longitudinal axis of system 100. Strands or ribbons 122 cooperate to provide an expansion framework that is controlled to be movable between a reduced-radius delivery contracted configuration and an enlarged-radius expanded configuration which will be explained in more detail below with respect to FIGS. 3-4. Open spaces 128 disposed between the plurality of strands or ribbons 122 when support structure 120 is expanded allow blood or other fluid to flow there through during the stapling procedure such that the blood vessel is not blocked or occluded. In addition, open spaces 128 are of a sufficient size such that the stapling device may be maneuvered through and between strands 122. One of ordinary skill in the art will appreciate that support structure 120 may include any number of strands or ribbons. For example, support structure 120 may include between two and eight strands or ribbons that extend generally parallel to the blood flow when expanded. Each of the plurality of strands or ribbons 122 has sufficient mechanical strength to expand at least a portion of graft 130 radially outward against a vessel wall of a body lumen. Strands or ribbons 122 are preferably constructed of implantable materials having good mechanical strength. For example, non-exhaustive examples of metallic materials for strands or ribbons 122 are stainless steel, cobalt based alloys (605L, MP35N), titanium, tantalum, superelastic nickel-titanium alloy, other biocompatible metals, thermoplastic polymers, or combinations of any of these.

Graft 130 is releasably attached to support structure 120 at connection 136 to ensure a secure mounting of graft 130 as it is tracked transluminally on system 100 to the target site. For example, in one embodiment, releasable sutures or thin strands of material (not shown) connect graft 130 to support structure 120. Connection 136 occurs generally at distal end 134 of graft 130 and approximately the middle of support structure 120 such that the graft material is pressed against the vessel wall when support structure 120 is expanded. Proximal end 132 of graft 130 is unattached to system 100. Connection 136 between graft 130 and support structure 120 is maintained until after a stapling device delivers one or more staples for attaching graft 130 to the target vessel. Once graft 130 is permanently secured to the vessel wall, the stapling device is removed and a separate cutting device is delivered within space 138 between outer shaft 106 and radially expanded graft 130 for cutting the sutures of connection 136 and thus disconnecting graft 130 from support structure 120. Support structure 120 may then be collapsed to the unexpanded condition and withdrawn. It will be apparent to those of ordinary skill in the art that graft 130 may be releasably attached to support structure 120 by any appropriate attachments means, such as releasable sutures described above. For example, in addition to or in the alternative, graft 130 may be held in frictional engagement with support structure 120 by the inclusion of slots, ridges, pockets, or other prosthesis retaining features formed into the exterior surface of outer shaft 106 to further ensure secure mounting of graft 130 as it is tracked transluminally on system 100 to the target site. The graft may be initially covered by an outer graft cover to keep the graft material compressed to gain access to the vasculature and travel to the treatment site. The outer graft cover can then be conventionally removed and the process proceed as described above.

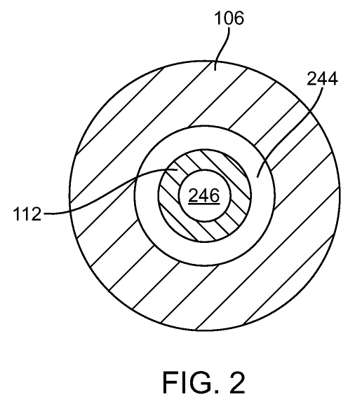

FIG. 2 is a cross-sectional view along line A-A of FIG. 1. As shown in FIG. 2, outer shaft 106 includes a lumen 244 extending there through. Inner shaft 112 extends through lumen 244 of outer shaft 106. Inner shaft 112 may include a guidewire lumen 246 defined by inner shaft 112 for receiving a guidewire (not shown) there through. When guidewire lumen 246 is present, inner shaft 112 may be advanced over an indwelling guidewire to track 100 to the target site. Alternatively, inner shaft 112 may instead be a solid rod (not shown) that does not have a lumen extending there through. In the solid rod embodiment, inner shaft 112 is tracked to the target site without the use of a guidewire with the assistance of tapered distal tip 118.

Outer and inner shafts 106, 112 may be formed of any suitable flexible polymeric material. Non-exhaustive examples of material for the catheter shafts are polyethylene terephalate (PET), nylon, polyethylene, PEBAX, or combinations of any of these, either blended or co-extruded. Optionally, a portion of the catheter shafts may be formed as a composite having a reinforcement material incorporated within a polymeric body to enhance strength, flexibility, and/or toughness. Suitable reinforcement layers include braiding, wire mesh layers, embedded axial wires, embedded helical or circumferential wires, and the like. In an embodiment, the proximal portions of the catheter shafts may in some instances be formed from a reinforced polymeric tube, for example, as shown and described in U.S. Pat. No. 5,827,242 to Follmer et al. which is incorporated by reference herein in its entirety. The catheter shafts may have any suitable working length, for example, 550 mm-600 mm, to extend to a target location where a staple is to be fired.

Referring now to FIGS. 3-4, support structure 120 is movable from an unexpanded configuration 340 (shown in FIG. 3) to an expanded configuration 450 (shown in FIG. 4). In unexpanded configuration 340, strands or ribbons 122 of support structure 120 are relatively straight to minimize the delivery profile as graft delivery system 100 is advanced to the target site. Support structure 120 is then radially expanded via distal advancement of outer shaft 106 (as indicated by directional arrow 452) to expanded configuration 450 shown in FIG. 4. In expanded configuration 450, strands or ribbons 122 of support structure 120 assume a balloon shaped cage which may approximate ellipsoid, spherical, and/or cylinder like shapes such that support structure 120 pushes graft 130 against the vessel wall. Support structure 120 is expanded to the balloon shaped cage which may approximate ellipsoid, spherical, and/or cylinder like shapes in situ to ensure graft 130 is pressed against the vessel wall until a stapling device delivers one or more staples for attaching graft 130 to the target vessel. Once graft 130 is permanently attached to the vessel wall via one or more staples, outer shaft 106 is retracted in a proximal direction (as indicated by directional arrow 342 in FIG. 3) to collapse support structure 120 back to unexpanded configuration 340. Once support structure 120 is radially collapsed, graft delivery system 100 may be retracted and withdrawn.

An actuator such as handle 109 shown in FIG. 1 may be provided at proximal portion 102 of graft delivery system 100 for moving outer shaft 106 and thus expanding support structure 120 to the balloon shaped cage which may approximate ellipsoid, spherical, and/or cylinder like expanded shapes. Handle 109 may be a push-pull actuator that is attached or connected to proximal end 108 of outer shaft 106 to expand support structure 120 such that when handle 109 is pushed, while holding proximal end 114 of inner shaft 112 fixed, outer shaft 106 is advanced in a distal direction to expand support structure 102. Alternatively, the actuator may be a turning knob (not shown) that is attached or connected to proximal end 108 of outer shaft 106 such that when the knob is rotated, outer shaft 106 is advanced in a distal direction to expand support structure 102. Thus, when the actuator is operated (i.e., manually turned or pushed), outer shaft 106 is distally advanced over inner shaft 112. Since distal end 126 of support structure 120 is fixed to inner shaft 112, support structure 120 radially expands or deploys to the expanded balloon shaped cage which may approximate ellipsoid, spherical, and/or cylinder like shapes. Although embodiments are described with outer shaft 106 being movable along inner shaft 112 to expand support structure 120, it should be apparent to one of ordinary skill in the art that support structure 120 may alternatively be expanded by proximally retracting inner shaft 112 while holding outer shaft 106 stationary. When inner shaft 112 is retracted in a proximal direction, support structure 120 radially expands or deploys to the expanded balloon shaped cage which may approximate ellipsoid, spherical, and/or cylinder like shapes since proximal end 124 of support structure 120 is fixed to outer shaft 106.

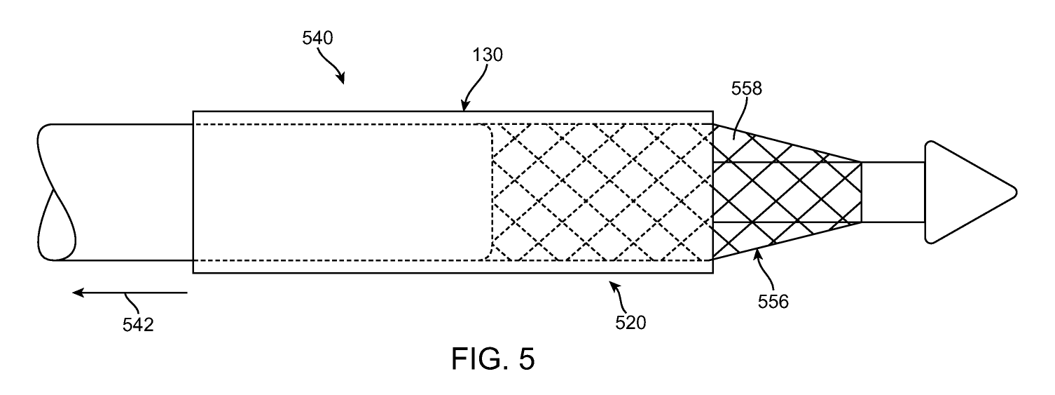

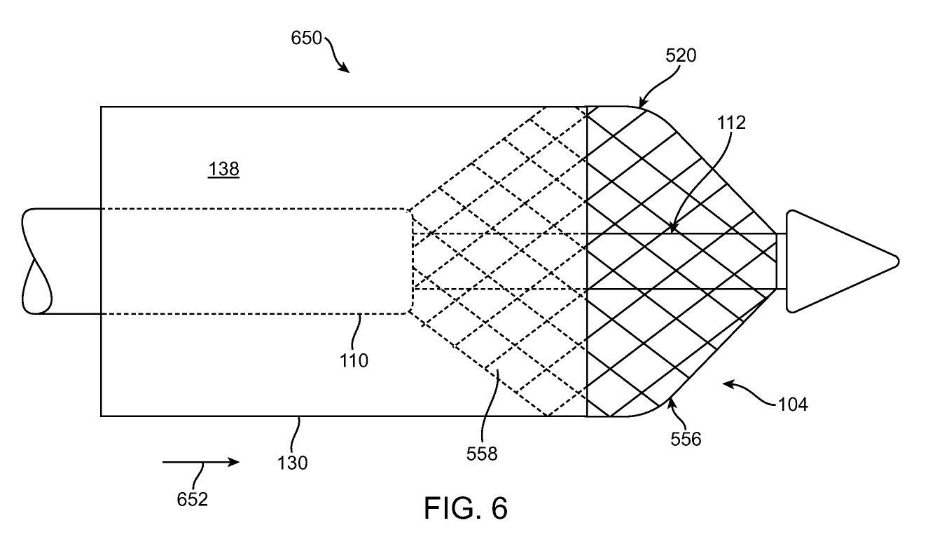

Another embodiment of an expandable support structure which may be utilized for securing a graft against a vessel wall is shown in FIGS. 5-6. Support structure 520 includes a braided structure or mesh 556. FIG. 5 illustrates support structure 520 in an unexpanded configuration 540, while FIG. 6 illustrates support structure 520 in an expanded configuration 650. Open spaces 558 disposed within mesh 556 when support structure 520 is expanded allow blood or other fluid to flow there through during the stapling procedure such that the blood vessel is not blocked or occluded. In addition, open spaces 558 are of a sufficient size such that the stapling device may be maneuvered through and/or between the elements of mesh 556. Mesh 556 provides uniform expansion of graft 130, and also holds the shape of graft 130 against the vessel wall until a stapling device delivers one or more staples for attaching graft 130 to the target vessel. The braided structure or mesh 556 has sufficient mechanical strength to press at least a portion of graft 130 to a vessel wall of a body lumen. Mesh 556 of support structure 520 is preferably constructed of implantable polymeric or metallic materials having good mechanical strength. Non-exhaustive examples of polymeric materials for mesh 556 are polyurethane, polyethylene terephalate (PET), nylon, polyethylene, PEBAX, or combinations of any of these, either blended or co-extruded. Non-exhaustive examples of metallic materials for mesh 556 are stainless steel, cobalt based alloys (605L, MP35N), titanium, tantalum, superelastic nickel-titanium alloy, or combinations of any of these.

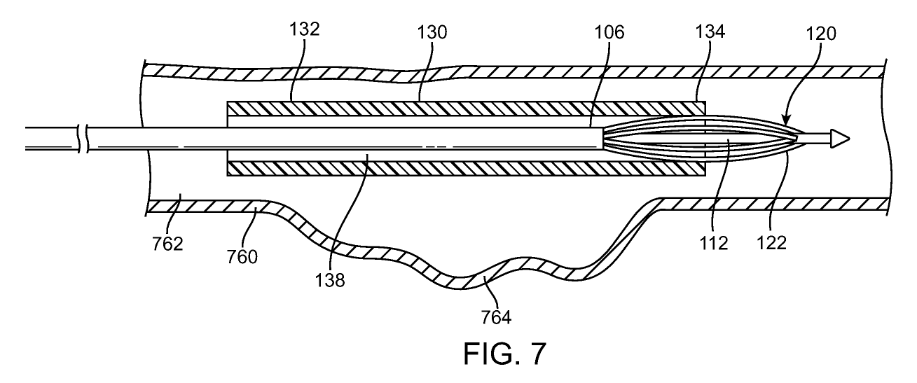

Referring now to FIGS. 7-14, a method of implanting graft 130 within an aneurysm 764 using graft delivery system 100 is described. FIG. 7 is a side view of graft delivery system 100 disposed within a target blood vessel 760 having a body lumen 762. Graft delivery system 100 is tracked to and properly positioned within vessel 760 such that graft 130 spans aneurysm 764. Support structure 120 having a plurality of parallel strands 122 is in the unexpanded straightened configuration as it is tracked to aneurysm 764. The graft system may include a graft cover (not shown) holding the graft in a compressed configuration as it is tracked and delivered. Distal end 134 of graft 130 is attached to support structure 120, and thus graft 130 is also in an unexpanded configuration.

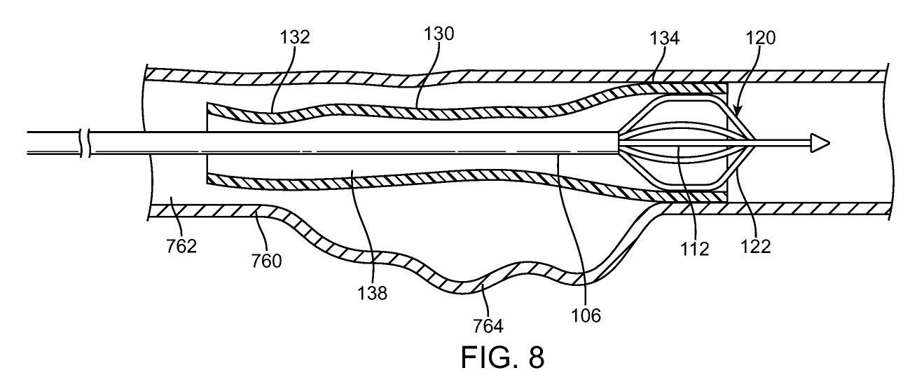

Once graft delivery system 100 is in place as desired, support structure 120 is radially expanded via distal advancement of outer shaft 106 to the expanded configuration shown in FIG. 8. In the expanded configuration, strands or ribbons 122 of support structure 120 assume a balloon shaped cage which may approximate ellipsoid, spherical, and/or cylinder like shapes such that support structure 120 pushes distal end 134 of graft 130 against vessel 760. Support structure 120 is expanded to the balloon shaped cage which may approximate ellipsoid, spherical, and/or cylinder like shapes in situ to press graft 130 against the vessel wall until a stapling device delivers one or more staples for attaching graft 130 to vessel 760 of body lumen 762. Deployment of distal end 134 of graft 130 at least partially deploys proximal end 132 of graft 130 such that the stapling device may be inserted through the partially expanded graft adjacent outer shaft 106. More particularly, once distal end 134 of graft 130 is radially expanded by support structure 120, blood flow enters the graft and at least partially expands proximal end 132 of graft 130 radially towards the vessel wall. Blood flow maintains the graft in at least a partially opened condition along the length of the graft.

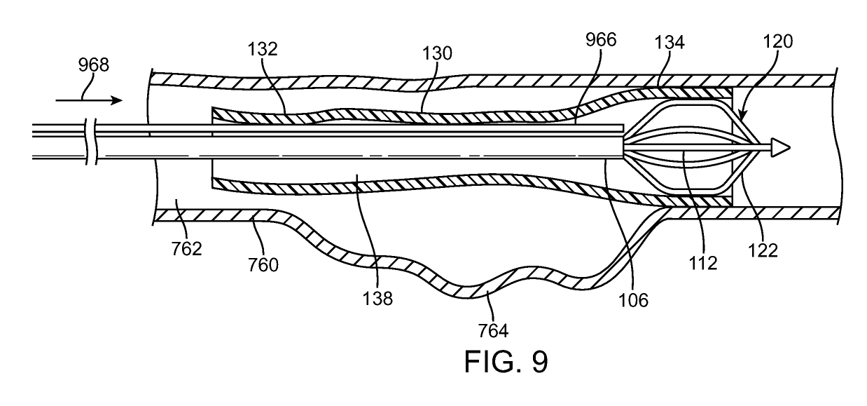

Referring now to FIG. 9, a stapling device 966 may be delivered within space 138 between outer shaft 106 and partially expanded graft 130. Stapling device 966 is distally advanced as indicated by directional arrow 968 until the distal end of stapling device 966 encounters support structure 120. When stapling device 966 encounters support structure 120, stapling device 966 is maneuvered through and between strands 122 within open spaces 128 so that stapling device 966 may be positioned as desired within or past support structure 120. Once stapling device 966 is in place (that is, adjacent a receiving area of graft 130 where a staple is to be fired), stapling device 966 according to its operation delivers one or more staples to permanently attach graft 130 to vessel 762. Expanded support structure 120 maintains graft 130 in a desired position against vessel 960 while multiple staples are delivered. In addition, when the staple is fired (or delivered) from stapling device 966, expanded support structure 120 prevents graft 130 from moving during the firing of the staple. Stapling device 966 is then moved to a second position in preparation for the firing of a second or subsequent staple, and the process is repeated to deploy subsequent staples.

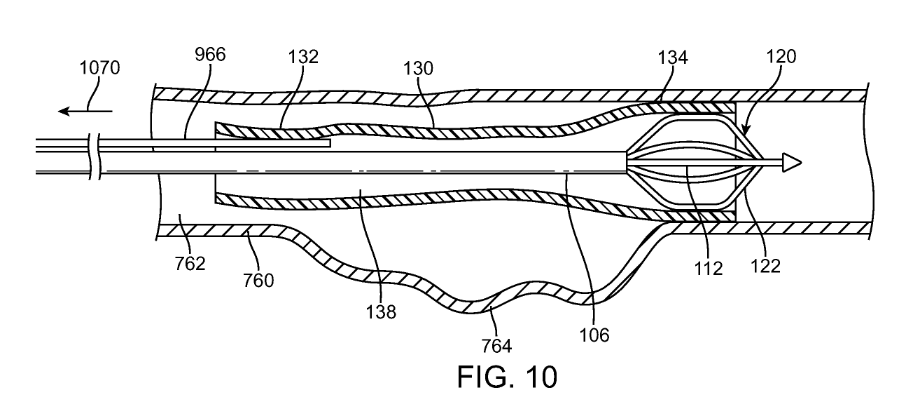

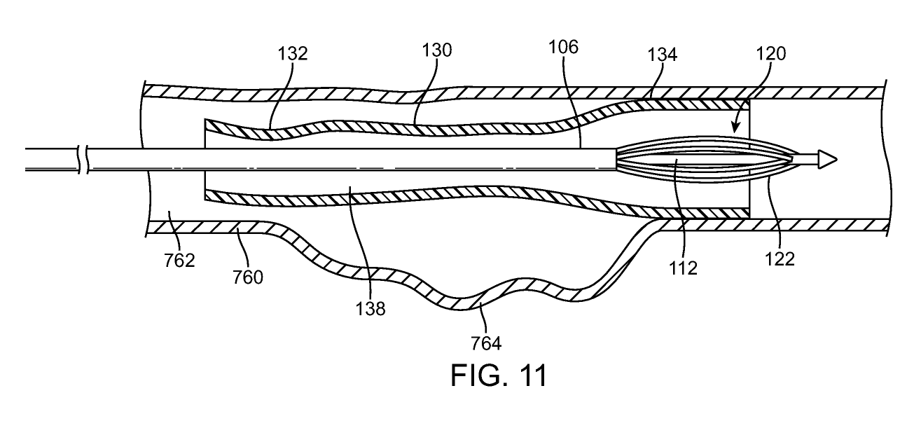

Once graft 130 is secured as desired, stapling device 966 is proximally retracted as indicated by directional arrow 1070 as shown in FIG. 10. Stapling device 966 is removed and a separate cutting device (not shown) is delivered within space 138 between outer shaft 106 and radially expanded graft 130 for disconnecting graft 130 from support structure 120. The separate cutting device cuts the sutures connecting graft 130 to support structure 120 as described above, and then is retracted and withdrawn. Where other methods of releasably attaching the graft material to the support device are utilized, such a tethered knots with pull strings, the release of those structures should be utilized. Stapling device 966 may include the cutting device such that the steps of removing the stapling device and inserting a separate cutting device may be consolidated. After support structure 120 is disconnected from expanded graft 130, support structure 120 may then be collapsed to the unexpanded straightened configuration shown in FIG. 11. To collapse support structure 120, outer shaft 106 is retracted in a proximal direction. Graft 130 remains in the expanded configuration, stapled to vessel 760. Once support structure 120 is radially collapsed, graft delivery system 100 may be retracted and removed from the patient.

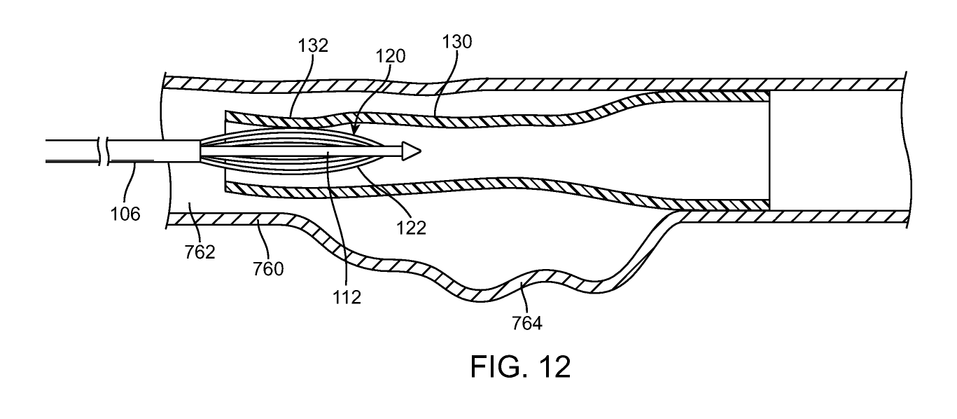

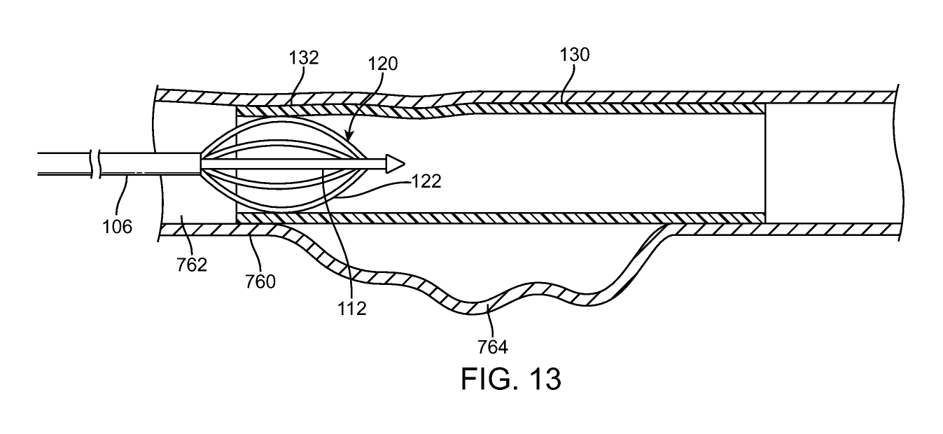

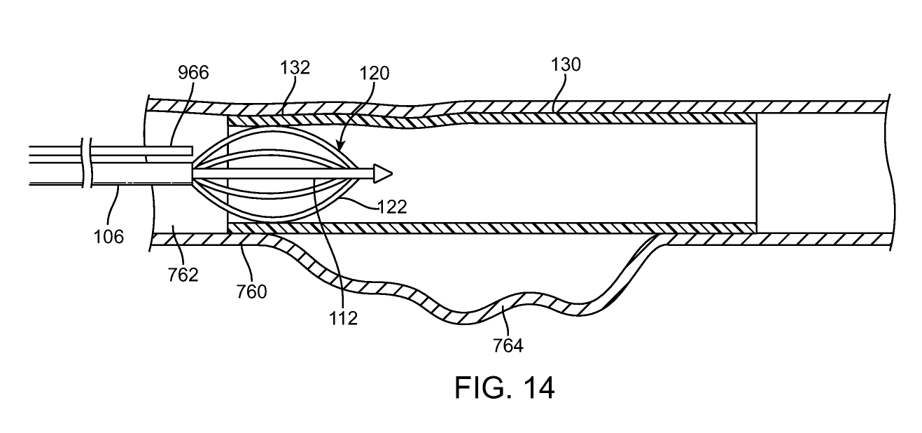

In one embodiment shown in FIGS. 12-14, it may be desirable to utilize the support structure to staple the proximal end 132 of graft 130 to vessel 760. More particularly, after distal end 134 of graft 130 is radially expanded by the support structure, outer shaft 106 may retracted in a proximal direction to collapse support structure 120 back to the unexpanded configuration. As shown in FIG. 12, graft delivery system 100 is further retracted such that the support structure is located within a proximal end 132 of graft 130. Once graft delivery system 100 is in place as desired, support structure 120 is then re-expanded to the balloon shaped cage which may approximate ellipsoid, spherical, and/or cylinder like shaped expanded configuration to fully expand the proximal end of graft 130 which may cause the central portion of the graft to expand as well as shown in FIG. 13. In the expanded configuration, strands or ribbons 122 of support structure 120 assume a balloon shaped cage which may approximate ellipsoid, spherical, and/or cylinder like shapes such that support structure 120 pushes proximal end 132 of graft 130 against vessel 760. By fully expanding proximal end 132 of graft 130, graft 130 may assume a fully opened condition along the length of the graft (see FIG. 13). Support structure 120 is expanded to the balloon shaped cage which may approximate ellipsoid, spherical, and/or cylinder like shapes in situ to press graft 130 against the vessel wall until a stapling device delivers one or more staples for attaching graft 130 to vessel 760 of body lumen 762. As shown in FIG. 14, stapling device 966 is then delivered between outer shaft 106 and fully expanded graft 130. Once stapling device 966 is in place (that is, adjacent a receiving area of graft 130 where a staple is to be fired), stapling device 966 delivers one or more staples to permanently attach proximal end 132 of graft 130 to vessel 760. Expanded support structure 120 maintains proximal end 132 of graft 130 in a desired position against vessel 960 while the staples are delivered. Stapling device 966 may be moved to a second position in preparation for the firing of a second or subsequent staple, and the process is repeated to deploy subsequent staples. Once graft 130 is secured as desired, stapling device 966 is proximally retracted and withdrawn.

In another embodiment of the present invention, it is not required to expand support structure 120 within the proximal end 132 of graft 130. Rather, deployment of distal end 134 of graft 130 may fully deploy proximal end 132 of graft 130 (not shown). Once distal end 134 of graft 130 is radially expanded by support structure 120, blood flow enters the graft and may expand proximal end 132 of graft 130 against the vessel wall. Blood flow may maintain the proximal portion of the graft in its fully opened condition in contact with the vessel walls along the length of the graft. While in its fully opened condition, stapling device 966 may be retracted to a position within proximal end 132 of graft 130 to secure proximal end 132 to vessel 760 if desired.

While various embodiments according to the present invention have been described above, it should be understood that they have been presented by way of illustration and example only, and not limitation. It will be apparent to persons skilled in the relevant art that various changes in form and detail can be made therein without departing from the spirit and scope described. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of delivering a tubular endoluminal graft through a body lumen, the method comprising the steps of:
tracking a graft delivery system to a target location within the body lumen, wherein the graft delivery system includes an expandable support structure at a distal portion of the system, wherein the graft is releasably attached to the support structure;
radially expanding the support structure to a balloon shaped cage expanded configuration such that at least a portion of the graft abuts against a vessel wall of the body lumen;
tracking a stapling device to the radially expanded graft such that the stapling device is adjacent to the target location within the body lumen;
delivering at least one staple from the stapling device to attach the graft to the vessel wall while the expanded support structure holds at least a portion of the graft against the vessel wall;
retracting the stapling device;
disconnecting the graft from the support structure;
radially contracting the support structure to a delivery configuration; and
retracting the graft delivery system with the support structure in the delivery configuration.

2. The method of claim 1, wherein a distal end of the graft is releasably attached to the support structure by means of sutures.

3. The method of claim 2, wherein the step of disconnecting the graft from the support structure includes tracking a cutting device to the expanded support structure and utilizing the cutting device to cut the sutures and disconnect the graft from the support structure.

4. The method of claim 1, wherein the graft delivery system includes an outer shaft and an inner shaft disposed within a lumen of the outer shaft, wherein a proximal end of the expandable support structure is connected to a distal end of the outer shaft and a distal end of the expandable support structure is connected to a distal end of the inner shaft.

5. The method of claim 4, wherein the stapling device is delivered through a space between the outer shaft of the graft delivery system and the graft.

6. The method of claim 4, wherein the step of radially expanding the support structure includes distally advancing the outer shaft over the inner shaft.

7. The method of claim 6, wherein the step of radially contracting the support structure includes proximally retracting the outer shaft over the inner shaft.

8. The method of claim 7, wherein the graft delivery system further includes an actuator for radially expanding and radially contracting the support structure, and wherein the actuator is selected from the group consisting of a push-pull actuator and a turning actuator.

9. The method of claim 4, wherein a distal end of the inner shaft is tapered.

10. The method of claim 1, wherein the support structure is formed from a plurality of strands having open spaces there between sufficient to allow the stapling device to maneuver through the support structure, the plurality of strands extending generally parallel to the blood flow.

11. The method of claim 1, wherein the support structure is formed from a mesh having open spaces sufficient to allow the stapling device to maneuver through the support structure.

12. The method of claim 1, wherein the expanded support structure prevents or reduces movement of the graft during firing of the staple.

13. The method of claim 1, wherein the step of radially expanding the support structure radially expands a distal end of the graft and thereafter blood flow through the graft expands a proximal end of the graft.

14. The method of claim 1, wherein the step of radially expanding the support structure radially expands a distal end of the graft, wherein after the step of radially contracting the support structure, the support structure is retracted proximally and re-expanded to expand a proximal end of the graft.

15. A method of delivering a tubular endoluminal graft through a body lumen, the method comprising the steps:
tracking a graft delivery system to a target location within the body lumen, wherein the graft delivery system includes an outer shaft, an inner shaft disposed within the lumen of the outer shaft, and an expandable support structure having a plurality of generally parallel strands with open spaces there between at a distal portion of the system, wherein a proximal end of the expandable support structure is connected to a distal end of the outer shaft, a distal end of the expandable support structure is connected to a distal end of the inner shaft, and the graft is releasably connected to the support structure;
distally advancing the outer shaft over the inner shaft to radially expand the support structure to a balloon shaped cage expanded configuration such that at least a portion of the graft abuts against a vessel wall of the body lumen;
tracking a stapling device through a space between the outer shaft and the graft;
tracking the stapling device through the open spaces of the support structure;
firing at least one staple from the stapling device to attach the graft to the vessel wall while the expanded support structure holds at least a portion of the graft against the vessel wall;
retracting the stapling device;
disconnecting the graft from the support structure;
proximally retracting the outer shaft over the inner shaft to radially contract the support structure to a straightened delivery configuration; and
retracting the graft delivery system with the support structure in the straightened delivery configuration.

16. The method of claim 15, wherein a distal end of the graft is releasably attached to the support structure by means of sutures and wherein the step of disconnecting the graft from the support structure includes tracking a cutting device to the expanded support structure and utilizing the cutting device to cut the sutures and disconnect the graft from the support structure.

17. A method of delivering a tubular endoluminal graft through a body lumen, the method comprising the steps:
tracking a graft delivery system to a target location within the body lumen, wherein the graft delivery system includes an outer shaft, an inner shaft disposed within the lumen of the outer shaft, and an expandable cage support structure at a distal portion of the system, wherein a proximal end of the expandable support structure is connected to a distal end of the outer shaft, a distal end of the expandable support structure is connected to a distal end of the inner shaft, and the graft is releasably connected to the support structure;
distally advancing the outer shaft over the inner shaft to radially expand the support structure to a balloon shaped cage expanded configuration such that at least a portion of the graft abuts against a vessel wall of the body lumen;
tracking a stapling device through a space between the outer shaft and the graft;
tracking the stapling device through open spaces of the cage support structure;
firing at least one staple from the stapling device to attach the graft to the vessel wall while the expanded support structure holds at least a portion of the graft against the vessel wall;
retracting the stapling device;
disconnecting the graft from the support structure;
proximally retracting the outer shaft over the inner shaft to radially contract the support structure to a straightened delivery configuration; and
retracting the graft delivery system with the support structure in the straightened delivery configuration.

18. The method of claim 17, wherein a distal end of the graft is releasably attached to the support structure by means of sutures and wherein the step of disconnecting the graft from the support structure includes tracking a cutting device to the expanded support structure and utilizing the cutting device to cut the sutures and disconnect the graft from the support structure.

19. A graft delivery system for delivering a tubular endoluminal graft through a body lumen, the system comprising:
an outer shaft;
an inner shaft disposed within the lumen of the outer shaft;
an expandable support structure at a distal portion of the system, wherein a proximal end of the expandable support structure is connected to a distal end of the outer shaft, a distal end of the expandable support structure is connected to a distal end of the inner shaft;
a graft releasably attached to the support structure, the graft having no stent structure coupled thereto; and
an actuator provided at a proximal portion of the system for moving the outer shaft relative to the inner shaft to expand the support structure to a configuration having open spaces sufficient to allow a stapling device to maneuver there through.

20. The system of claim 19, wherein the support structure is formed from a plurality of strands extending generally parallel to the blood flow.

21. The system of claim 19, wherein the support structure is formed from a mesh.
